(19) United States
(12) Patent Application Publication
(10) Pub. No.: US 2004/0006111 A1
(43) Pub. Date: Jan. 8, 2004

(54) Transmucosal delivery of proton pump inhibitors

(76) Inventors: Kenneth Widder, Rancho Santa Fe, CA (US); Warren Hall, San Diego, CA (US); Kay Olmstead, San Diego, CA (US)

Correspondence Address: Richard H. Pagliery, Brobeck, Phleger & Harrison LLP, 12390 El Camino Real, San Diego, CA, 92130-2081, US

(21) Appl. No.: 10/353,143

(22) Filed: Jan. 27, 2003

Related U.S. Application Data
(60) Provisional application No. 60/351,909, filed on Jan. 25, 2002.
(60) Provisional application No. 60/374,761, filed on Apr. 22, 2002.

(51) Int. Cl. A61K 31/4439; A61K 9/24

(52) U.S. Cl. 514/338; 424/471

(57) ABSTRACT

The present invention relates to pharmaceutical compositions and methods for transmucosal delivery of proton pump inhibitors. In one embodiment, the pharmaceutical composition of the present invention comprises a core which comprises an antacid, and an outer layer surrounding the core. The outer layer contains a therapeutically effective amount of a proton pump inhibitor. In another embodiment, the pharmaceutical composition of the present invention comprises an outer layer which comprising a unidirectional film, and an inner layer which contains a therapeutically effective amount of a proton pump inhibitor. In yet another embodiment, the pharmaceutical composition of the present invention is a unidirectional tablet for delivery of a proton pump inhibitor across the oral mucosa. In this embodiment, the pharmaceutical composition contains an outer layer which contains a pharmaceutically acceptable water impermeable layer, and an inner layer which contains a therapeutically effective amount of a proton pump inhibitor.

### RELATED APPLICATION

The present application claims the benefit of priority to U.S. Provisional Application No. 60/351,909, filed on Jan. 25, 2002 and U.S. Provisional Application No. 60/374,761, filed on Apr. 22, 2002 and are incorporated by reference in their entirety.

### FIELD OF THE INVENTION

The present invention relates to the field of gastrointestinal pharmacology. In particular, compositions and methods for transmucosal delivery of substituted benzimidazole proton pump inhibitors are described.

### BACKGROUND OF THE INVENTION

Proton pump inhibitors, also know as gastric H+/K+ inhibitors, are potent suppressors of gastric acid secretion. Over the past decade, they have been found to be the most effective drugs in antiulcer therapy (Goodman & Gilman's The Pharmacological Basis of Therapeutics (Joel G. Hardman et al. eds., 2001)). Currently available for clinical use are proton pump inhibitors such as omeprazole (PRILOSEC®), lansoprazole (PREVACID®), rabeprazole (ACIPHEX®)), pantoprazole (PROTONIX®) and esomeprazole (NEXIUM®). These proton pump inhibitors are a-pyridylmethylsulfinyl benzimidazoles with different substitutions on the pyridine or the benzimidazole groups.

Proton pump inhibitors are prodrugs that require activation in an acidic environment. Upon parietal cell entry, these prodrugs are activated by a proton-catalyzed process that results in the formation of a thiophilic sulfenamide or sulfenic acid. It is this activated form that reacts by covalent binding with the sulfhydryl group of cysteins from the extracellular domain of the H+/H+ ATPase to irreversibly inhibit gastric acid production.

Proton pump inhibitors are unstable at low pH and thus are typically supplied as enteric-coated granules encapsulated in a gelatin capsule (omeprazole, esomeprazole, and lansoprazole), as enteric-coated tablets (pantoprazole and rabeprazole), or as multiple pellet systems (esomeprazole-MUPS, omeprazole-MUPS). The enteric coating dissolves only upon exposure to a neutral to mildly alkaline pH, thus preventing degradation of the drugs by acid in the esophagus and stomach. Once absorbed from the small intestines, proton pump inhibitors are extensively metabolized in the liver by the cytochrome P450 system.

Therefore, besides having a delayed onset of action between one to four hours or more, enteric-coated formulations have poor bioavailability. Bioavailability is further decreased if the drug is taken with food due to delayed gastric emptying. Thus, enteric-coated proton pump inhibitor formulations currently on the market are generally taken prior to meals or on an empty stomach.

New dosage formats are being developed to enhance administration to patients who have difficulty taking standard tablets or capsules. U.S. Pat. No. 6,328,994 describes new dosage formats that are taken with or without the use of water. However, the microgranules used in these disintegrable tablets are enteric-coated to provide acid resistance and are designed to be absorbed in the intestine and not absorbed by the oral mucosal surface. U.S. Pat. No. 6,489,346 describes a pharmaceutical composition which is not enteric-coated, comprising a proton pump inhibitor and a buffering agent in the amount of 0.1 mEq to approximately 2.5 mEq per mg of proton pump inhibitor wherein the dosage form is selected from a suspension tablet, chewable tablet, effervescent powder, and effervescent tablet.

Alternative routes of administration are being explored to improve oral proton pump inhibitor bioavailability. Bioadhesive pharmaceutical formulations can be used to deliver drugs systemically through absorption from the site of application. One primary requirement for this type of delivery is that an effective concentration of the particular pharmaceutical be maintained at the site for a long enough period of time to allow for sufficient absorption for systemic effects.

Bioadhesive formulations are known in the art and include gels, pastes, tablets, and films. For example, U.S. Pat. Nos. 5,192,802; 5,314,915; 5,298,258; and 5,642,749 describe bioadhesive gels. Denture adhesive pastes are described in, for example, U.S. Pat. Nos. 4,894,232 and 4,518,721. A commercial product under the name Orabase, which is a thick gel or paste for the relief or mouth sores, is another example of an adhesive paste. Bioadhesive tablets are described in U.S. Pat. Nos. 4,915,948; 4,226,848; 4,292,299; and 4,250,163, as having single layer or bilayers.

The use of bandages or bioadhesive laminated films, which are thinner and flexible and therefore have decrease foreign body sensation, are described in U.S. Pat. Nos. 3,996,934 and 4,286,592. U.S. Pat. Nos. 6,159,498 and 5,800,832 describe bioerodable, water-soluble adhesives which are capable of adhering to mucosal surfaces for localized delivery. These products are used to deliver drugs through the skin or mucous. The laminated films usually include an adhesive layer and a backing layer with or without an intermediate reservoir layer.

In addition to film systems for the delivery of drug through the skin, film delivery systems for use on mucosal surfaces are also described. These types of systems, which are water-insoluble and usually in the form of laminated, extruded, or composite films, are described in U.S. Pat. Nos. 4,517,173 (describing a membrane-adhering film consisting of at least three layers, including a pharmaceutical layer containing a drug and a cellulose derivative selected from hydroxyropyl cellulose, methyl cellulose, and hydroxypropyl methyl cellulose; a poor water soluble layer made from a combination of one or more cellulose derivatives with a poor water soluble fatty acid; and an intermediate layer made of cellulose derivatives); 4,572,832 (describes a soft film for buccal delivery, made by the combined use of a water soluble protein, a polyol, and a polyhydric alcohol such as cellulose and polysaccharides and teaches the use of coloring or flavoring agents); 4,713,243 (describes a single or multi-layered bioadhesive thin film made from 40-95% water soluble hydroxypropyl cellulose, 5-60% water-insoluble ethylene oxide, 0-10% water-insoluble ethyl cellulose, propyl cellulose, polyethylene, or polypropylene, and a medicament. The films are three layered laminates and include a bioadhesive layer, a reservoir layer, and a non water-soluble outer protective layer); 4,900,554 (describes a soft adhesive film applicable to the oral mucosa containing a systemic drug and comprising a mixture of vinyl acetate non water-soluble homopolymer, an acrylic acid polymer, and a cellulose derivative); and 5,137,729 (describes a device for use in the oral cavity having an adhesive layer including a mixture of an acrylic acid polymer, a water-insoluble cellulose derivative, and a pharmaceutical preparation, and a water-insoluble or sparingly soluble backing layer). The adhesive layer in the '729 patent contains the pharmaceutical and, upon application to the mucosal surface, delivers the drug.

A bioerodable film for mucosal delivery is also described in the art. U.S. Pat. Nos. 6,159,498 and 5,800,832 describe a biodegradable water-soluble film which comprises a flexible film having a first water-soluble adhesive layer, a second water-soluble non-adhesive layer, and a pharmaceutical composition. The second water-soluble non-adhesive backing layer comprises hydroxyethyl cellulose. Both the '958 and '832 patents describe the delivery of pharmaceuticals in the therapeutic areas of anti-inflammatory analgesic agents, steroidal anti-inflammatory agents, antihistamines, local anesthetics, bactericides and disinfectants, basoconstrictors, hemostatics, chemotherapeutic drugs, antibiotics, keratolytics, cauterizing agents, and antiviral drugs. The first water-soluble adhesive layer comprises hydroxyethyl cellulose, polyacrylic acid, and sodium carboxymethyl cellulose wherein the pharmaceutical composition is incorporated into one of the water-soluble layers.

An adhesive tablet that delivers omeprazole by absorption through the buccal mucosa was described in Choi et al., Development of Omeprazole Buccal Adhesive Tablets with Stability Enhancement in Human Saliva, J. Control. Rel. 68:397-404 (2000) and Choi et al., Formulation and In Vivo Evaluation of Omeprazole Buccal Adhesive Tablet, J. Control. Rel. 68:405-412 (2000). The buccal adhesive tablets described in each of these articles were composed of sodium alginate, hydroxypropylmethylcellulose (HPMC), magnesium oxide and croscarmellose sodium and prepared by compressing all of the ingredients together using a Erweka tablet machine (Frankfrut, Germany). As shown by the data, omeprazole release from the buccal tablets was relatively slow, taking 45 minutes to generate peak plasma concentration of 370 ng/ml. This formulation also exhibited low bioavailability.

The disclosures of the references cited herein are hereby incorporated by reference in their entirety.

### SUMMARY OF THE INVENTION

The present invention is directed to a pharmaceutical composition for delivery of a proton pump inhibitor across an oral mucosal surface. In one embodiment, the pharmaceutical composition of the present invention comprises a core which comprises an antacid, and an outer layer surrounding the core. The outer layer contains a therapeutically effective amount of a proton pump inhibitor. In another embodiment, the pharmaceutical composition of the present invention comprises an outer layer which comprises a unidirectional film, and an inner layer which contains a therapeutically effective amount of a proton pump inhibitor. In yet another embodiment, the pharmaceutical composition of the present invention is a unidirectional tablet for buccal delivery of a proton pump inhibitor. In this embodiment, the pharmaceutical composition contains an outer layer which contains a pharmaceutically acceptable water impermeable layer, and an inner layer which contains a therapeutically effective amount of a proton pump inhibitor.

### BRIEF DESCRIPTION OF THE DRAWINGS

FIG. 1 shows a side, cross-sectional view of a tablet having an inner core which contains antacid and an outer layer that contains a proton pump inhibitor.

FIG. 2 shows a side view of a buccal patch having an inner layer which contains a bioadhesive material and a proton pump inhibitor, an outer layer which contains a unidirectional film, and an optional wax coating over the outer layer.

FIG. 3 shows a side view of a buccal tablet having an inner layer which contains a proton pump inhibitor and an outer layer which contains a unidirectional film.

### DETAILED DESCRIPTION OF THE INVENTION

As used herein, the terms “comprising,” “including,” and “such as” are used in their open, non-limiting sense.

The term “bioerodable” means that the component, carrier, or formulation erodes, over time, in biological media such as bodily fluids and anatomical structures comprising or bathed by body fluids. Examples of bodily fluids include blood, plasma, saliva, tears, lymph, urine, etc. Examples of anatomical structures comprising or bathed by bodily fluids include the oral cavity, the nasal cavity, the genitourinary. tract, the respiratory tract, the gastrointestinal tract, etc. Such erosion in bodily fluids may be due to factors such as dissolution, dispersion, friction, gravity, etc. The terms water-erodable and bioerodable are used interchangeably.

The term “prodrug” as used herein refers to a compound that is converted under physiological conditions or by solvolysis or metabolically to a specified compound that is pharmaceutically active, wherein the precursor may or may not be pharmaceutically active. Prodrugs of a compound may be routinely identified using techniques known in the art. See, e.g., Bertolini et al., J. Med. Chem. (1997), 40:2011-2016; Shan et al., J. Pharm. Sci. (1997), 86 (7):765-767; Bagshawe, Drug Dev. Res. (1995), 34:220-230; Bodor, Advances in Drug Res. (1984), 13:224-331; Bundgaard, Design of Prodrugs (Elsevier Press 1985); Larsen, Design and Application of Prodrugs, Drug Design and Development (Krogsgaard-Larsen et al. eds., Harwood Academic Publishers, 1991); Dear et al., J. Chromatogr. B (2000), 748:281-293; Spraul et al., J. Pharmaceutical & Biomedical Analysis (1992), 10 (8):601-605; and Prox et al., Xenobiol. (1992), 3 (2):103-112.

The term “pharmaceutically acceptable salt” refers to a salt that retains the biological effectiveness of the free acid and/or base of the specified compound. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phylacetates, phenylpropionates, phenylbutyrates, citrates, lactates, gamma-hydroxybutyrates, glycollates, tartarates, methane-sulfonates, propanesulfonates, naphthalene-1-sulfonates, naphthalene-2-sulfonates, and mandelates. Several of the officially approved salts are listed in Remington: The Science and Practice of Pharmacy, Ch. 38, Mack Publ. Co., Easton (19^{th} Ed., 1995).

If an inventive compound is a base, a desired salt may be prepared by any suitable method known to the art, including treatment of the free base with an inorganic acid such as hydrochloric acid, hydrobromic acid, sulfuric acid, nitric acid, phosphoric acid, and the like; or with an organic acid such as acetic acid, maleic acid, succinic acid, mandelic acid, fumaric acid, malonic acid, pyruvic acid, oxalic acid, glycolic acid, salicylic acid, a pyranosidyl acid such as glucuronic acid or galacturonic acid, an alpha-hydroxy acid such as citric acid or tartaric acid, an amino acid such as aspartic acid or glutamic acid, an aromatic acid such as benzoic acid or cinnamic acid, a sulfonic acid such as p-toluenesulfonic acid or ethanesulfonic acid; or the like.

If an inventive compound is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary amines; secondary amines; ; tertiary amines; and cyclic amines such as piperidine, morpholine, and piperazine; as well as inorganic salts derived from sodium, calcium, potassium, magnesium, manganese, iron, copper, zinc, aluminum, and lithium.

In the case of compounds, salts, or solvates that are solids, it is understood by those skilled in the art that the inventive compounds, salts, and solvates may exist in different crystal forms, all of which are intended to be within the scope of the present invention and specified formulas. Pharmaceutical compounds may exist as single geometric isomers, stereoisomers, racemates, and/or mixtures of enantiomers and/or diastereomers. All such single geometric isomers, stereoisomers, racemates, and mixtures thereof are intended to be within the broad scope of the present invention.

A “derivative” of a compound means a chemically modified compound wherein the chemical modification takes place at one or more functional groups of the compound and/or on an aromatic ring, when present. The derivative however, is expected to retain the pharmacological activity of the compound from which it is derived.

Examples of “solvates” suitable for the present invention include compounds of the invention in combination with water, isopropanol, ethanol, methanol, DMSO, ethyl acetate, acetic acid, or ethanolamine.

A “therapeutically effective amount” is intended to mean, consistent with considerations known in the art, an amount of a pharmaceutical agent effective to achieve a pharmacological effect or therapeutic improvement without undue adverse side effects In the case of proton pump inhibitors, a therapeutically effective amount may be, for example, an amount that provides a level of parietal cell activation and/or H+/H+ ATPase inhibition that is recognized in the art to be therapeutically effective.

A “proton pump inhibitor” or “PPI” refers to any substituted benzimidazole possessing pharmacological activity as an inhibitor of H+/H+ ATPase. Examples of PPIs suitable to be used in this invention include omeprazole, hydroxyomeprazole, esomeprazole, lansoprazole, pantoprazole, rabeprazole, dontoprazole, habeprazole, perprazole (s-omeprazole magnesium), ransoprazole, pariprazole, and leminoprazole in neutral form, as well as the pharmaceutically acceptable salt, prodrug, derivative, enantiomer, isomer, free base, anhydrate, hydrate, solvate, polymorph or combinations thereof, whether in crystalline form, amorphous form or a combination thereof, of such proton pump inhibitor.

Examples of “antacids” suitable for the present invention include alkaline earth metal salts and bicarbonate salts of a Group IA metals. Illustrative examples of salts useful in the present invention include sodium bicarbonate, potassium bicarbonate, magnesium hydroxide, magnesium lactate, magnesium gluconate, magnesium oxide, magnesium carbonate, magnesium silicate, other magnesium salts, aluminum hydroxide, aluminum hydroxide/sodium bicarbonate coprecipitate, aluminum glycinate, sodium citrate, sodium tartarate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium gluconate, an acid salt of an amino acid, an alkali salt of an amino acid, or combinations thereof.

A “unidirectional film” is designed to allow for substantially one sided delivery of a proton pump inhibitor across the oral mucosa. It substantially prevents delivery of a proton pump inhibitor across the film.

The term “water impermeable layer” as used in this invention includes any film, coating or other substrate that substantially prevents delivery of PPI across such layer.

A “multiple compressed tablet” is a tablet prepared by subjecting the fill material to more than a single compression.

Examples of “excipients” suitable for the present invention include acacia, alginic acid, croscarmellose, gelatin, gelatin hydrosylate, mannitol, plasdone, sodium starch glycolate, sorbitol, sucrose, and xylitol. Specifically for molded or compressed tablet formulations, suitable excipients that may be used include amorphous lactose, beta lactose, microcrystalline cellulose, croscarmellose sodium, dicalcium phosphate, carboxymethyl cellulose, hydroxypropyl cellulose, polyethylene gylcols, sodium lauryl sulfate, and the like.

Examples of “bioadhesive polymers” used in the present invention include, for example, alkyl celluloses, polysaccharides, polypeptides, synthetic polymers and mixtures thereof.

“Synthetic polymers” that may be used as bioadhesive polymers include, for example, vinyl and acrylic derivatives of carbomer, polycarbophil, polyethylene glycol, polyethylene oxide, polymethacrylates, polyvinyl alcohol, polyvinylpyrrolidone, and the like

“Alkyl celluloses” that may be used as bioadhesive polymers include, hydroxyethyl cellulose, hydroxypropyl cellulose, hydroxypropylmethyl cellulose, methyl cellulose, sodium carboxymethyl cellulose, calcium carboxymethyl cellulose, and the like.

“Polysaccharides” that may be used as bioadhesive polymers include, for example, acacia, agar, alginic acid and salts of alginic acid, carageenan, dextran, guar gum, karaya gum, pectin, tragacanth, xanthan gum, and the like.

Examples of “binders” suitable for the present invention include acacia, alginic acid, ethylcellulose, methylcellulose, microcrystalline cellulose, a derivatized cellulose, such as carboxymethyl cellulose, sodium carboxymethyl cellulose, hydroxyethyl cellulose, hydroxypropyl methylcellulose, and hydroxypropyl cellulose, dextrin, gelatin, glucose, guar gum, hydrogenated vegetable oil, type I, polyethylene glycol, lactose, compressible sugars, sorbitol, mannitol, dicalcium phosphate dihydrate, tricalcium phosphate, calcium sulfate dihydrate, maltodextrins, lactitol, magnesium carbonate, xylitol, magnesium aluminium silicate, maltodextrin, methylcellulose, hydroxypropylcellulose, polyethylene polyethylene oxide, polymethacrylates, povidone (polyvinylpyrrolidone), Plasdone, sodium alginate, starch, pregelatinized starch, and zein.

Examples of “lubricants” suitable for the present invention include magnesium stearate, stearic acid and its pharmaceutically acceptable alkali metal salts, calcium stearate, sodium stearate, Cab-O-Sil, Syloid, sodium lauryl sulfate, sodium chloride, magnesium lauryl sulfate, and talc.

“Polypeptides” that may be uses as bioadhesive polymers include, for example, casein, gelatin, protamine sulfate, and the like.

Examples of “permeation enhancers” suitable for this invention include medium chain triglycerides; bile salts; anionic surfactants such as docusate sodium and sodium lauryl sulfate; cationic surfactants such as benzalkonium chloride, benzethonium chloride, and cetrimide; non-ionic surfactants such as glyceryl monooleate, polyoxyethylene sorbitan fatty acid esters, polyvinyl alcohol, and sorbitan esters; alcohol(s); isopropyl myristate; oleic acid; and the like.

Examples of “solubility enhancers” suitable for the present invention include buffers, cosolvents, surfactants, and complexants such as polyamidoamine starburst dendrimers and cyclodextrins.

“Rapidly dispersing agents” suitable for the present invention include, for example, wicking agents (agents that transport moisture into the interior of a dosage form so that the dosage form can dissolve from the inside as well as from the outside), non-effervescent disintegrants, and effervescent disintegrants.

The term “wicking agents” as used in the present invention includes various non-effervescent disintegration agents such as microcrystalline cellulose; croscarmellose sodium; crosslinked polyvinylpyrrolidone; starches such as corn and potato starches, and modified starches; alginates; gums such as agar, arabic, guar, locust bean, karaya, pectin, and tragacanth; Carbopol®; hydroxyalkyl cellulose, hydroxypropylmethyl cellulose and the like. Wicking agents also include effervescent disintegration agents including compounds which evolve gas. The effervescent agents typically evolve gas by means of chemical reactions that occur upon exposure of the effervescent disintegration agent to saliva. The gas generating reaction is usually the result of a reaction between a soluble acid source and an alkaline metal carbonate or carbonate source that generates carbon dioxide gas upon contact with the water in saliva. The acid sources that may be used in the effervescent agent are any which are safe for human consumption, for example, food acids, and hydrite antacids such as citric, tartaric, malic, fumaric, adipic, succinic acid, and the like. Carbonate sources include dry solid carbonate and bicarbonate salts such as sodium bicarbonate, sodium carbonate, potassium bicarbonate, potassium carbonate, calcium carbonate, magnesium carbonate, and the like.

“Flavorants” suitable for use in the present invention include, for example, sucrose, sucralose, polyols such as xylitol and maltitol, sodium saccharide, Asulfame-K, Neotame® (Nutrasweet Co.), glycyrrhizin, malt syrup, citric acid, tartaric acid, menthol, lemon oil, citrus flavor, common salt, and other flavors known in the art.

The terms “stabilizers” or “preservatives” as used in the present invention include, for example parahydroxybenzoic acid alkyl esters, antioxidants, antifungal agents, and other stabilizers/preservatives known in the art.

A “coloring agent” as used in the present invention includes, for example, water soluble dye, Lake dye, ion oxide, natural colors, titanium oxide, and the like.

As described above, the bioavailability of a proton pump inhibitor after oral administration is generally low due to the degradation upon exposure to the acidic conditions of the stomach and hepatic fist pass metabolism. Transmucosal delivery of proton pump inhibitors provides an alternative route of administration that avoids gastric and hepatic degradative processes, thereby rapidly increasing plasma levels of these drugs. The present invention provides novel pharmaceutical compositions of proton pump inhibitors for transmucosal delivery. The pharmaceutical composition may be formulated for application and absorption across the palate, buccal, sublingual, or gingival mucosa.

Proton pump inhibitors that may be used include any substituted benzimidazole. Typically the proton pump inhibitor is selected from omeprazole, hydroxyomeprazole, esomeprazole, lansoprazole, pantoprazole, rabeprazole, dontoprazole, habeprazole, perprazole (s-omeprazole magnesium), ransoprazole, pariprazole, and leminoprazole in neutral form, as well as the pharmaceutically acceptable salt, prodrug, derivative, enantiomer, isomer, free base, anhydrate, hydrate, solvate, polymorph or combinations thereof, whether in crystalline form, amorphous form or a combination thereof, of such proton pump inhibitor. The proton pump inhibitor may be in a dosage form such as a powder, tablet, microspheres, or enteric-coated granules.

The antacid can be any alkaline earth metal salt, a bicarbonate salt of a Group IA metal, or a mixture thereof. Illustrative examples of salts useful in the present invention include sodium bicarbonate, potassium bicarbonate, magnesium hydroxide, magnesium lactate, magnesium gluconate, magnesium oxide, magnesium carbonate, magnesium silicate, other magnesium salts, aluminum hydroxide, aluminum hydroxide/sodium bicarbonate coprecipitate, aluminum glycinate, sodium citrate, sodium tartarate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium gluconate, an acid salt of an amino acid, an alkali salt of an amino acid, or combinations thereof.. In various embodiments of the invention the pharmaceutical compositions may include less than 50 mEq antacid, less than 25 mEq antacid, less than 10 mEq antacid, or less than 1 mEq antacid.

Variations of the present invention may also include flavorants, sweetening agents, absorption enhancers, mucoadhesive agents, or rapidly dispersing agents. Suitable absorption enhancers may include permeation enhancers and solubility enhancers. Rapidly dispersing agents that may be used include wicking agents, non-effervescent disintegrants, and effervescent disintegrants.

The inventive pharmaceutical composition may be formed as a partitioned tablet, e.g., a bi-layered tablet, or a multiple compressed tablet that is made by compressing a dosage form including a proton pump inhibitor around a compressed antacid core, or a bi-layer unidirectional film, patch or tablet. However, other oral solid dosage forms such as single compressed tablets or molded tablets may be used.

In use, the pharmaceutical composition may be applied to the intraoral mucosa, e.g., the buccal sublingual, gingival mucosa, or the palate. In one embodiment, the coating or layer of non-enteric-coated proton pump inhibitor disperses and the proton pump inhibitor is absorbed into the bloodstream. In other embodiments the inner layer of the bi-layer unidirectional film or tablet contains the proton pump inhibitor which is absorbed across the intraoral mucosa and into the bloodstream. The proton pump inhibitor then suppresses acid production at the gastric proton pumps. In a further embodiment, a resultant core containing an antacid or layer containing an antacid is then chewed or swallowed to provide heartburn relief.

### Outer Layer Containing a Proton Pump Inhibitor Around a Core Containing an Antacid

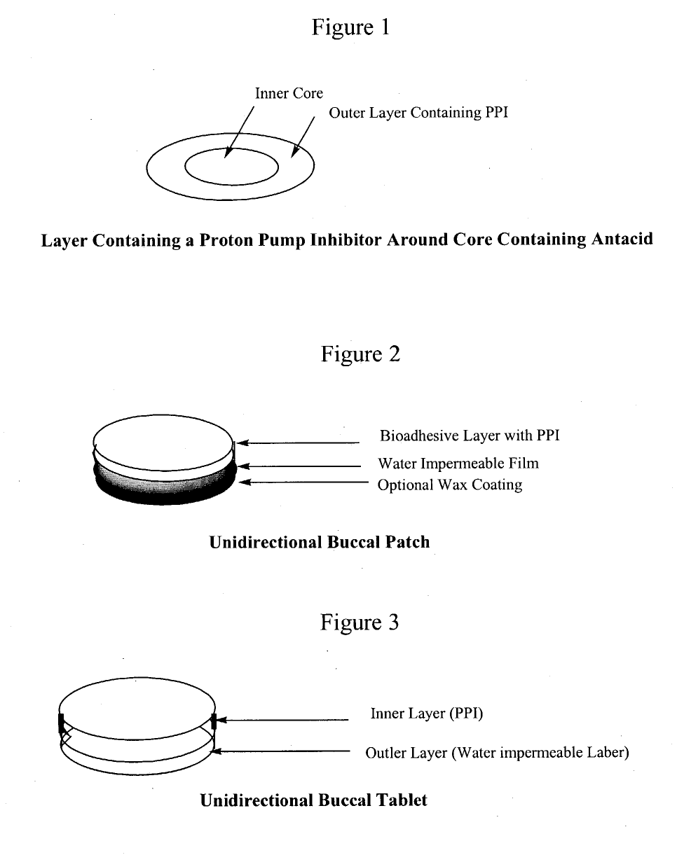

In one embodiment of the invention, as shown in FIG. 1, the antacid is contained in a core surrounded by an outer layer containing a PPI.

Outer Layer Around the Core Containing an Antacid

The outer layer around the core containing an antacid is designed to deliver a therapeutically effective amount of a PPI by absorption through the oral mucosa. The remaining antacid core is then left intact until chewed or swallowed.

The amount of PPI included in the formulation may be any amount that is therapeutically effective. For example, the amount of PPI included in the formulation may be between 5-150 mg. In some embodiments of the present invention, the amount of PPI in the formulation is between 5-150 mg, 10-80 mg, or 10-40 mg. For veterinary applications, the amount of PPI in the formulation may be that amount sufficient to provide from 1-10 mg or 2-5 mg of PPI per kg of body weight. Thus, a formulation intended for administration to a horse may contain, for example, from 0.5 gin to 10 gm, 0.5 gm to 5 gm, or from 0.5 to 3 gm. of PPI.

The PPI may be in the form of a powder, micronized powder, microspheres, microgranules, or other solid form.

Additionally, the rapidly dispersing PPI layer around the inner core containing an antacid may contain one or more of the following: a rapidly dispersing agent, a second pharmaceutical, an excipient, a flavorant, a stabilizer, a coloring agent, a binder, a filler, a diluent or other component related to formulation.

Core Containing an Antacid

Depending on the particular formulation and application, the amount of antacid in the pharmaceutical composition will vary. In one embodiment, the amount of antacid incorporated into the core may range from 1-60 mEq. In another embodiment the amount of antacid present in the core may range from 3-40 mEq. In veterinary applications, the amount of antacid may range from 1-1000 mEq, 1-500 mEq, or 1-100 mEq.

In contrast to most commercial formulations of PPIs that use an antacid or buffering agent to stabilize the PPI, one embodiment of the present invention contains a pharmaceutical composition that includes an antacid to provide relief from symptoms of acidpeptic disorders, e.g., heartburn, after a therapeutically effective amount of the PPI has been administered. Although an antacid is typically used in the core, other pharmaceutically active agents may be substituted in its place. In one embodiment, the antacid core is formulated as a chewable tablet.

In another embodiment, the core containing an antacid and the layer containing the PPI can be separated by a film or coating to provide a tactile sense that the PPI has been dissolved and that the antacid is ready to be chewed or swallowed. The film/coating may comprise, for example, a sugar coat, polymeric film, or any other tablet coating known in the art.

In addition to the above, the core containing an antacid or layer containing an antacid may contain one or more of the following: a rapidly dispersing agent, a second pharmaceutical, an excipient, a flavorant, a stabilizer, a coloring agent, a binder, a filler, a diluent or other component related to formulation.

### Bi-Layer Unidirectional Buccal Film

In another embodiment of the invention, as shown by FIG. 2, the bi-layer unidirectional buccal film may be comprised of a unidirectional outer layer and a. bioadhesive inner layer which contains the drug.

Outer Layer Containing Unidirectional Film The outer layer may be made of pharmaceutically accepted polymeric materials which are water impermeable and do not swell in contact with moisture, such as polyethylene, polyurethane, Mylar and the like.

The outer layer may also contain an absorbable gelatin film (Gelfilm®, Pharmacia Upjohn) as a flexible biocrodable backer layer.

Additionally, the outer layer may be coated with a waxy material to form a thin film. The waxy material may be used to prevent the PPI from being released into the oral cavity which results in the unidirectional release of the drug into the oral- mucosa. Pharmaceutical grade wax such as Camauba wax, Bees wax, Shea Butter, Candelilla, Glyceryl Behenate, and Camauba derivatives may be used to impart this water impermeability in the outer layer. In one embodiment, a low melting wax is chosen to avoid high temperature processing conditions, since most PPI's are thermally unstable. In another embodiment, the waxy material is Camauba wax.

Additionally, the outer layer may contain one or more of the following: an excipient, a flavorant, a stabilizer, a coloring agent, or other component related to formulation.

Inner Layer Containing Proton Pump Inhibitor

The inner layer of the bi-layer film includes at least one bioadhesive polymer and a PPI. The PPI is incorporated into the inner layer by either a pre-load or a post-load process. In one embodiment, permeation enhancers and/or solubility enhancers may be employed to assist the rate of transmucosal delivery. The solubility of PPI may be improved by complexation with Cyclodextrin (alpha-, beta-, gamma-, or substituted Cyclodextrin). This complexation can be done either as a discrete step prior to the formulation or during the drug loading step.

The amount of PPI included in the formulation may be any amount that is therapeutically effective. For example, the amount of PPI included in the formulation may be between 5-150 mg. In one embodiment, the amount of PPI in the formulation may be between 10-80 mg. In an alternative embodiment, the amount of PPI in the formulation may be between 10-40 mg. For veterinary applications, the amount of PPI in the formulation may be that amount sufficient to provide from 1-10 mg or 2-5 mg of PPI per kg of body weight. Thus, a formulation intended for administration to a horse may contain, for example, from 0.5 gm to 5 gm of PPI.

The PPI may be in the form of a powder, micronized powder, microspheres, microgranules, or other solid form.

Additionally, the inner layer may contain one or more of the following: a rapidly dispersing agent, a bioadhesive, a second pharmaceutical, an excipient, a flavorant, a stabilizer, a coloring agent, or other component related to formulation.

### Bi-Layer Unidirectional Buccal Tablet

In a further embodiment of the invention, as shown by FIG. 3, the bi-layer unidirectional buccal tablet contains a proton pump inhibitor in the inner layer and a outer layer comprising a waxy material which prevents the PPI from being released into the oral cavity, resulting in the unidirectional release of the PPI into the oral mucosa.

Outer Layer Containing Wax

The waxy material present in the outer layer of the bi-layer unidirectional tablet is a pharmaceutical grade wax. Examples of pharmaceutical grade waxes suitable for the present invention include Carnauba wax, Bees wax, Shea Butter, Candelilla, Glyceryl Behenate, and Camauba derivatives. In one embodiment, the waxy material is glyceryl behenate (Compitrol 888, Gattefosse).

In a further embodiment, the waxy layer aids in the compressibility of the outer layer in addition to providing water impermeability. The waxy layer may protect the PPI from the slightly acidic environment of the mouth, thereby eliminating the need for an alkaline component in the formulation of the inner layer.

Additionally, the outer layer may contain one or more of the following: an excipient, a flavorant, a stabilizer, a coloring agent, a binder, a filler, a diluent or other component related to formulation.

Inner Layer Containing Proton Pump Inhibitor

The inner layer may include at least one bioadhesive polymer and a PPI. The amount of PPI included in the formulation may be any amount that is therapeutically effective. For example, the amount of PPI included in the formulation may be between 5-150 mg. In one embodiment, the amount of PPI in the formulation maybe between 10-80 mg. In an alternative embodiment, the amount of PPI in the formulation may be between 10-40 mg. For veterinary applications, the amount of PPI in the formulation may be that amount sufficient to provide from 1-10 mg or 2-5 mg of PPI per kg of body weight. Thus, a formulation intended for administration to a horse may contain, for example, from 0.5 gm to 5 gm of PPI.

The PPI may be in the form of a powder, micronized powder, microspheres, microgranules, or other solid form.

In one embodiment of the invention, the inner layer also includes an antacid. The antacid may protect the PPI from degradation in the acidic environment of saliva or maintain product shelf-life of the pharmaceutical composition. Thus, both the amount of antacid and the antacid itself will be determined from the objective of its use. For example, less antacid may be necessary if the purpose is to maintain shelf life than if the purpose is to maintain stability of the PPI in saliva.

In another embodiment, magnesium carbonate is used. Magnesium carbonate may act as both an antacid and a binder. For pharmaceutical compositions applied directly to the buccal mucosa, it may be desirable to use a lesser amount of antacid, e.g., less than 1 mEq, less than 0.5 mEq, or less than 0.1 mEq, to keep the size of the dosage form manageable with respect to mucosal adhesiveness and mobility.

In another embodiment, hydroxypropyl cellulose (HPC) is used as a bioadhesive component. HPC has a long disintegration time, which may increase the time available for delivery by keeping the tablet from collapsing.

In a further embodiment, the bitter taste often associated with a PPI such as Omeprazole, may be masked by the addition of a flavorant. For example, direct compression grade xylitol (Xylitab 100 by Roquet) may impart a pleasing taste and mouth feel for the application duration.

In one embodiment, the inner layer contains a lubricant, for example, stearic acid or magnesium stearate.

In another embodiment of the invention, the antacid is provided as a layer adjacent to the PPI layer, e.g., as with a film.

Additionally, the inner layer may contain one or more of the following: a rapidly dispersing agent such as a wicking agent, a bioadhesive, a second pharmaceutical, an excipient, a flavorant, a stabilizer, a coloring agent, a binder, a filler, a diluent or other component related to formulation.

### Methods of Formulation

The pharmaceutical compositions of the present invention may be formulated as partitioned tablets, films, or any other solid, semi-solid, gel, or paste oral dosage form known in the art. For example, the pharmaceutical composition can be a molded or compressed tablet which may include one or more binder, diluent, adhesive, wicking agent, absorption enhancer such as a permeability enhancer and/or a solubility enhancer, lubricant, flavorant, or coloring agent.

In one embodiment, the pharmaceutical composition is formed by selecting a PPI dosage form and compressing the PPI dosage around the core containing an antacid. In another embodiment, the PPI is in the dosage form of a micronized powder.

In a further embodiment, a layered tablet or film is formed by configuring the layered tablet or film to have an inner layer to be in contact with the oral mucosal surface and an outer layer surface to allow for substantially one-sided delivery of the PPI across the oral mucosa.

In other embodiments of the present invention, the pharmaceutical composition are prepared by techniques widely known in the art such as wet or dry granulation, direct compression, or molding.

### Methods of Administration

In contrast to various PPI formulations currently in commercial use, the pharmaceutical compositions embodied in the present invention may provide the option of on-demand usage by the patient because the pharmaceutical compositions of this invention may be taken on an empty stomach or after a meal, allow for more rapid absorption of the PPI into the bloodstream, and, if desired, contain an antacid. For example, the pharmaceutical composition can be placed on an oral mucosal surface such as the sublingual mucosa, buccal mucosa, gingiva, or palate where the PPI is absorbed.

In one embodiment, the PPI may be absorbed through the oral mucosa into the bloodstream. In further embodiments, a therapeutically effective amount of the PPI is absorbed within 60 minutes, within 30 minutes, or within 15 minutes after placing it on the oral mucosa.

In another embodiment, the PPI is absorbed leaving a core containing an antacid or a layer containing an antacid each of which may provide heartburn relief when the patient chews or swallows the core containing the antacid or the layer containing the antacid.

In various embodiments, the pharmaceutical composition may be used for the treatment or prevention of gastric acid disorders including, but not limited to, gastric or duodenal ulcers, gastroesophageal reflux disease, severe erosive esophagitis, and pathological hypersecretory conditions such as Zollinger-Ellision Syndrome. Treatment of these conditions and/or symptoms of these conditions may be accomplished by administering to a patient a pharmaceutically effective amount of the pharmaceutical composition according to the present invention.

The invention has been described by the physical and pharmaceutical properties and benefits of the formulation. This manner of describing the invention, should not, however, be taken as limiting the scope of the invention in any way.

The following specific examples are provided solely to illustrate particular representative embodiments of the invention. Accordingly, the following examples should not be construed as limiting the scope of the invention in any way.

### EXAMPLE 1

Core Containing Antacid With PPI Coating

Inner Core Containing Antacid

| Starting Material | mg/tablet | % of Composition |
|---|---|---|
| Calcium Carbonate-95S (Destab) | 1053.3 | 77.9% |
| Hydroxypropyl Cellulose | 55 | 4.1% |
| Xylitab 100 | 200 | 14.8% |
| Flavor/Sweetener | 30 | 2.2% |
| Magnesium Stearate | 13 | 1.0% |
| Total Inner Core Containing Antacid | 1351.3 | 100% |

Half of the total calcium carbonate-95S, hydroxypropyl cellulose, flavor/sweetener, xylitab 100 and then the remaining half of the direct compression grade calcium carbonate-95S are placed in a sequential manner into a suitable blender through a sifter equipped with an appropriate screen. The mixture is blended until homogeneous. Alternatively, the hydroxypropyl cellulose, flavor/sweetener are pre-blended with xylitab 100 to facilitate their passage through the sifter. The mixture is then screened into the blender through a #30 mesh screen and the magnesium stearate is added. The mixture is then blended for 2-5 minutes to lubricate the blend.

Outer Layer

| Starting Material | mg/tablet | % of composition |
|---|---|---|
| Omeprazole | 40 | 6.5% |
| Calcium Carbonate-95S (Destab) | 50 | 7.7% |
| Xylitab 100 | 450 | 69.6% |
| Microcrystalline Cellulose | 70 | 10.8% |
| Croscarmellose Sodium | 30 | 4.6% |
| Magnesium Stearate | 7 | 1.1% |
| Total Outer Core | 647 | 100% |

Omeprazole is blended with Calcium Carbonate-95S. The mixture is then placed a suitable blender through a sifter equipped with screen. Microcrystalline cellulose, croscarmellose sodium, xylitab 100, and the omeprazole/calcium carbonate pre-mixture are then blended until the mixture becomes homogeneous. The mixture is then screened into the blender through a #30 mesh screen and the magnesium stearate is added. The mixture is then blended for 2-5 minutes to lubricate the blend.

Compression Coating (Dry Coating or Press Coating)

Using tabletting equipment specifically designed for the purpose of compression coating, the outer layer blend is placed into a tablet hopper designed for this purpose. The inner core containing antacid blend is then placed into its respective tablet hopper. During press coating, one turret contains the dye and punches used to product the inner core containing antacid. The inner antacid core blend is then picked up by a transfer system and carried to a second turret containing dies and punches that product the final tablet image. In these dies, a “bed” of outer layer material is deposited. The cores are placed into these dies on the “bed” of the outer layer material. As the turret rotates, the final portion of outer coating is deposited into the dies containing the cores. The material in these dies is then compressed which consolidates the outer layer material around the inner antacid core to product the final compression coated tablet.

### EXAMPLE 2

Bi-layer Unidirectional Buccal Patch

### Example 2(a)—Pre-loading Omeprazole in Bi-layer Film

Polyurethane film sheet is coated in one side with melted Carnauba wax (Koster Keunen, Inc.) at 70-80° C. for 1-2 seconds. The thin wax coating on the film is allowed to cool to dryness at room temperature.

The bioadhesive gel is prepared by mixing Polycarbophil (Noveon AA1, BF Goodrich) in ethanol. The dispersion is stirred until a homogeneous viscous gel results. The required amount of polyacrylic acid (Carbopol 934, BF Goodrich) is added to the dispersion while stirring at high speed. After the addition of ethanol to the required weight, the viscous gel is slowly stirred in a closed container at an ambient temperature. Micronized Omeprazole powder is added to the viscous gel while stirring. Once a homogeneous gel is obtained, the required weight of gel is slowly casted into the wax-coated polyurethane film sheet by pouring at a steady state speed.

The total weight of the gel casted pre sheet is pre-determined by correlation of gel thickness/weight gain per area of the sheet. This results in the final bi-layer film containing 10+/−0.2 mg of total omeprazole per 8-inch disc. Ethanol is completely removed by gentle movement of an air dryer over the casted film until a constant weight is achieved. The circular or oblong bi-layer films are punched from the larger films and stored at room temperature away from the light.

### Example 2(b)—Pre-loading Omeprazole in Bi-layer Film with Cyclodextrin as a Solubility Enhancer

Polyethylene film sheet is coated in one side with melted Carnauba wax (Koster Keunen, Inc.) at 70-80° C. for 1-2 seconds. The thin wax coating on the films is allowed to cool to dryness at ambient condition. The coating will harden within 5 seconds and cooled to room temperature.

Bioadhesive gel is prepared by mixing Polycarbophil (Noveon AA1, BF Goodrich) in ethanol. The dispersion was stirred until a homogeneous viscous gel is formed. The required amount of polyacrylic acid (Carbopol 934, BF Goodrich) is added to the dispersion while stirring the mixture at a high speed. After the addition of ethanol to the required weight, the viscous gel is slowly stirred in a closed container at an ambient temperature. Gamma cyclodextrin-Omeprazole complex, the preparation method of which is well known in the art, is then added to the gel while stirring the viscous gel at ambient condition. See, e.g., EP 0991407. Once the homogeneous gel is obtained, the required weight of gel is slowly casted into the wax-coated polyurethane film sheet by pouring at a steady state speed.

The total weight of gel casted per sheet is pre-determined by correlation of gel thickness/weight gain per area of the sheet. This will result in the final bi-layer film containing 10+/−0.2 mg of total Omeprazole per ⅜″ disc. Ethanol is completely removed by gentle movement of air dryer over the casted film until a constant weight is achieved. The circular or oblong bi-layer films (⅜ inch diameter) are punched from the larger films and stored in ambient conditions away from light.

### EXAMPLE 3

Bi-layer Unidirectional Buccal Tablet

Outer Layer

| Starting Material | mg/tablet |
|---|---|
| Klucel EXP (HPC) | 10 |
| Dicalcium Phosphate | 10 |
| Destab Magnesium Carbonate-90S | 20 |
| FD & C Lake Red No. 40 | 0.1 |
| Glyceryl Behenate (Compitol 888) | 2 |
| Total Weight of the Outer Layer | 42.2 |

The outer layer powder is prepared by mixing Klucel EXP (HPC), MgCO3, Destab Magnesium Carbonate-90S, FD & C Lake Red No. 40, and Glyceryl Behenate (Compitol 888).

Inner Layer

| Starting Material | mg/tablet |
|---|---|
| Omeprazole, USP or its salt equivalent | 20 |
| Destab Magnesium Carbonate-90 S | 20 |
| Klucel EXP (HPC) | 6 |
| Xylitab 100 | 10 |
| Magnesium Stearate | 0.6 |
| Total of Inner Layer | 56.6 |

Omeprazole or its salt form is pre-mixed with Magnesium Carbonate-90S for a short time (about 3-5 minutes) in an appropriate sized blender followed by addition of HPC and Xylitab 100. The mixture is then subjected to additional mixing to form a homogeneous blend. Magnesium Stearate is then added to the blend and the mixture is blended for an additional 2-5 minutes.

Compression of the Bi-layer Tablet

The bi-layer tablet is compressed using a double-sided rotary tablet press equipped with dual hoppers; one containing the outer layer blend and the second containing the inner layer blend.

The invention is described and depicted above with respect to particular illustrative embodiments. However, alternative embodiments exist which do not depart from the scope and spirit of the invention. Accordingly, the scope of the invention encompasses the following claims and their legal equivalents and is not limited to the embodiments discussed and depicted above.

What is claimed is:

1. A pharmaceutical composition comprising:
a core comprising an antacid; and
an outer layer surrounding the core, said outer layer comprising a therapeutically effective amount of a proton pump inhibitor, or a pharmaceutically acceptable salt, prodrug, derivative, enantiomer, free base, isomer, polymorph, hydrate, anhydrate or solvate thereof.

2. The pharmaceutical composition of claim 1, wherein the proton pump inhibitor is selected from the group of omeprazole, hydroxyomeprazole, esomeprazole, lansoprazole, pantoprazole, rabeprazole, dontoprazole, habeprazole, perprazole, ransoprazole, pariprazole, leminoprazole, and pharmaceutically acceptable salts, prodrugs, derivatives, enantiomers, free bases, isomers, polymorphs, hydrates, anhydrates and solvates thereof.

3. The pharmaceutical composition of claim 2, wherein the proton pump inhibitor is omeprazole or a pharmaceutically acceptable salt, prodrug, derivative, enantiomer, free base, isomer, polymorph, hydrate, anhydrate or solvate thereof.

4. The pharmaceutical composition of claim 2, wherein the proton pump inhibitor is lansoparazole, rabeprazole, pantoprazole, or esomeprazole, or a pharmaceutically acceptable salt, prodrug, derivative, enantiomer, free base, isomer, polymorph, hydrate, anhydrate or solvate thereof.

5. The pharmaceutical composition of claim 1, wherein the outer layer comprises 5-150 mg of the proton pump inhibitor.

6. The pharmaceutical composition of claim 5, wherein the outer layer comprises 10-80 mg of the proton pump inhibitor.

7. The pharmaceutical composition of claim 6, wherein the outer layer comprises 10-40 mg of the proton pump inhibitor.

8. The pharmaceutical composition of claim 1, wherein the outer layer comprises 0.5-10 grams of the proton pump inhibitor.

9. The pharmaceutical composition of claim 8, wherein the outer layer comprises 1-3 grams of the proton pump inhibitor.

10. The pharmaceutical composition of claim 1, wherein the outer layer further comprises an excipient.

11. The pharmaceutical composition of claim 1, wherein the core further comprises an excipient.

12. The pharmaceutical composition of claim 1, wherein the outer layer further comprises an antacid.

13. The pharmaceutical composition of claim 1, wherein the antacid is an alkaline metal salt, a bicarbonate salt of a Group IA metal, or a combination thereof.

14. The pharmaceutical composition of claim 13, wherein the antacid is magnesium carbonate or calcium carbonate.

15. The pharmaceutical composition of claim 13, wherein the antacid is sodium bicarbonate or potassium bicarbonate.

16. The pharmaceutical composition of claim 1, wherein the outer layer further comprises a solubility enhancer.

17. The pharmaceutical composition of claim 16, wherein the solubility enhancer is cyclodextrin.

18. The pharmaceutical composition of claim 1, wherein the outer layer further comprises a rapidly dispersing agent selected from the group of wicking agents, non-effervescent disintegrants, and effervescent disintegrants.

19. The pharmaceutical composition of claim 18, wherein the rapidly dispersing agent is croscarmellose sodium.

20. The pharmaceutical composition of claim 1, wherein upon oral administration of the composition to a mammal, a therapeutically effective amount of the proton pump inhibitor is absorbed across the oral mucosal surface in less than 1 hour and the antacid core remains substantially intact until chewed or swallowed.

21. The pharmaceutical composition of claim 20, wherein upon oral administration of the composition to a mammal, a therapeutically effective amount of the proton pump inhibitor is absorbed across the oral mucosal surface in less than 45 minutes and the antacid core remains substantially intact until chewed or swallowed.

22. The pharmaceutical composition of claim 21, wherein upon oral administration of the composition to a mammal, a therapeutically effective amount of the proton pump inhibitor is absorbed across the oral mucosal surface in less than 30 minutes and the core remains substantially intact until chewed or swallowed.

23. The pharmaceutical composition of claim 22, wherein upon oral administration of the composition to a mammal, a therapeutically effective amount of the proton pump inhibitor is absorbed across the oral mucosal surface in less than 15 minutes and the core remains substantially intact until chewed or swallowed.

24. The pharmaceutical composition of claim 1, wherein the proton pump inhibitor is in the form of a powder, microspheres, micronized powder, or non-enteric coated microgranules.

25. A pharmaceutical composition suitable for oral mucosal delivery of a proton pump inhibitor to a mammal, comprising:
an outer layer comprising a unidirectional film; and
an inner layer comprising a therapeutically effective amount of a proton pump inhibitor or a pharmaceutically acceptable salt, prodrug, derivative, enantiomer, free base, isomer, polymorph, hydrate, anhydrate or solvate thereof.

26. The pharmaceutical composition of claim 25, wherein the proton pump inhibitor is selected from the group of omeprazole, hydroxyomeprazole, esomeprazole, lansoprazole, pantoprazole, rabeprazole, dontoprazole, habeprazole, perprazole, ransoprazole, pariprazole, leminoprazole, and pharmaceutically acceptable salts, prodrugs, derivatives, enantiomers, free bases, isomers, polymorphs, hydrates, anhydrates and solvates thereof.

27. The pharmaceutical composition of claim 26, wherein the proton pump inhibitor is omeprazole or a pharmaceutically acceptable salt, prodrug, derivative, enantiomer, free base, isomer, polymorph, hydrate, anhydrate or solvate thereof.

28. The pharmaceutical composition of claim 26, wherein the proton pump inhibitor is lansoparazole, rabeprazole, pantoprazole, or esomeprazole or a pharmaceutically acceptable salt, prodrug, derivative, enantiomer, free base, isomer, polymorph, hydrate, anhydrate or solvate thereof.

29. The pharmaceutical composition of claim 25, wherein the outer layer comprises 0.5-10 grams of the proton pump inhibitor.

30. The pharmaceutical composition of claim 29, wherein the outer layer comprises 1-3 grams of the proton pump inhibitor.

31. The pharmaceutical composition of claim 25, wherein the outer layer comprises 5-150 mg of the proton pump inhibitor.

32. The pharmaceutical composition of claim 31, wherein the outer layer comprises 10-80 mg of the proton pump inhibitor.

33. The pharmaceutical composition of claim 32, wherein the outer layer comprises 10-40 mg of the proton pump inhibitor.

34. The pharmaceutical composition of claim 25, wherein upon oral administration of the composition to a mammal, a therapeutically effective amount of the proton pump inhibitor is absorbed across the oral mucosal surface in less than 2 hours.

35. The pharmaceutical composition of claim 34, wherein upon oral administration of the composition to a mammal, a therapeutically effective amount of the proton pump inhibitor is absorbed across the oral mucosal surface in less than 1 hour.

36. The pharmaceutical composition of claim 35, wherein upon oral administration of the composition to a mammal, a therapeutically effective amount of the proton pump inhibitor is absorbed across the oral mucosal surface in less than 45 minutes.

37. The pharmaceutical composition of claim 36, wherein upon oral administration of the composition to a mammal, a therapeutically effective amount of the proton pump inhibitor is absorbed across the oral mucosal surface in less than 30 minutes.

38. The pharmaceutical composition of claim 37, wherein upon oral administration of the composition to a mammal, a therapeutically effective amount of the proton pump inhibitor is absorbed across the oral mucosal surface in less than 15 minutes.

39. The pharmaceutical composition of claim 25, wherein the outer layer comprises a pharmaceutically acceptable polymer selected from the group of polyethylene, polyurethane, Mylar and mixtures thereof.

40. The pharmaceutical composition of claim 39, wherein the pharmaceutically acceptable polymer is polyurethane.

41. The pharmaceutical composition of claim 25, wherein the unidirectional film is absorbable or bioerodable.

42. The pharmaceutical composition of claim 41, wherein the unidirectional film comprises Gelfilm.

43. The pharmaceutical composition of claim 25, further comprising a pharmaceutically acceptable water impermeable layer covering the outer layer.

44. The pharmaceutical composition of claim 43, wherein the water impermeable layer comprises a waxy material.

45. The pharmaceutical composition of claim 44, wherein the waxy material is selected from the group of Camauba wax, Bees wax, Shea Butter, Candelilla, Glyceryl Behenate, and Camauba derivatives and mixtures thereof.

46. The pharmaceutical composition of claim 45, wherein the waxy material is Camauba wax.

47. The pharmaceutical composition of claim 25, further comprising a flavorant.

48. The pharmaceutical composition of claim 25, further comprising a coloring agent.

49. The pharmaceutical composition of claim 25, wherein the inner layer further comprises a bioadhesive material.

50. The pharmaceutical composition of claim 49, wherein the bioadhesive material comprises a bioadhesive polymer selected from the group of an alkyl cellulose, hydroxypropyl cellulose, a polysaccharide, a polypeptide, a synthetic polymer and mixtures thereof.

51. The pharmaceutical composition of claim 50, wherein the bioadhesive polymer is an alkyl cellulose, hydroxypropyl cellulose or a polysaccharide.

52. The pharmaceutical composition of claim 25, wherein the proton pump inhibitor is in the form of a powder, microspheres, micronized powder, or non-enteric coated microgranules.

53. The pharmaceutical composition of claim 52, wherein the proton pump inhibitor is in the form of micronized powder.

54. A unidirectional tablet for transmucosal delivery of a proton pump inhibitor to a mammal, comprising:
an outer layer comprising a pharmaceutically acceptable water impermeable layer; and
an inner layer comprising a therapeutically effective amount of a proton pump inhibitor or a pharmaceutically acceptable salt, prodrug, derivative, enantiomer, free base, isomer, polymorph, hydrate, anhydrate or solvate thereof.

55. The pharmaceutical composition of claim 54, wherein the proton pump inhibitor is selected from the group of omeprazole, hydroxyomeprazole, esomeprazole, lansoprazole, pantoprazole, rabeprazole, dontoprazole, habeprazole, perprazole, ransoprazole, pariprazole, leminoprazole, and pharmaceutically acceptable salts, prodrugs, derivatives, enantiomers, free bases, isomers, polymorphs, hydrates, anhydrates and solvates thereof.

56. The pharmaceutical composition of claim 55, wherein the proton pump inhibitor is omeprazole or a pharmaceutically acceptable salt, prodrug, derivative, enantiomer, free base, isomer, polymorph, hydrate, anhydrate or solvate thereof.

57. The pharmaceutical composition of claim 55, wherein the proton pump inhibitor is lansoparazole, rabeprazole, pantoprazole, or esomeprazole or a pharmaceutically acceptable salt, prodrug, derivative, enantiomer, free base, isomer, polymorph, hydrate, anhydrate or solvate thereof.

58. The pharmaceutical composition of claim 54, wherein the water impermeable layer comprises a waxy material.

59. The pharmaceutical composition of claim 58, wherein the waxy material is selected from the group of Camauba wax, Bees wax, Shea Butter, Candelilla, Glyceryl Behenate, and Carnauba derivatives and mixtures thereof.

60. The pharmaceutical composition of claim 59, wherein the waxy material is Camauba wax.

61. The pharmaceutical composition of claim 54, wherein the inner layer further comprises an antacid.

62. The pharmaceutical composition of claim 61, wherein the antacid is magnesium carbonate.

63. The pharmaceutical composition of claim 54, wherein the outer layer comprises 0.5-10 grams of the proton pump inhibitor.

64. The pharmaceutical composition of claim 63, wherein the outer layer comprises 1-3 grams of the proton pump inhibitor.

65. The pharmaceutical composition of claim 54 wherein the outer layer comprises 5-150 mg of the proton pump inhibitor.

66. The pharmaceutical composition of claim 65, wherein the outer layer comprises 10-80 mg of the proton pump inhibitor.

67. The pharmaceutical composition of claim 66, wherein the outer layer comprises 10-40 mg of the proton pump inhibitor.

68. The pharmaceutical composition of claim 54, wherein the inner layer further comprises a binder.

69. The pharmaceutical composition of claim 68, wherein the binder is magnesium carbonate.

70. The pharmaceutical composition of claim 54, wherein the inner layer further comprises a bioadhesive material.

71. The pharmaceutical composition of claim 54, further comprising a bioadhesive layer in contact with the outer surface of the inner layer.

72. The pharmaceutical composition of claim 71, wherein the bioadhesive material is hydroxypropyl cellulose.

73. The pharmaceutical composition of claim 54, wherein the inner layer further comprises a solubility enhancer.

74. The pharmaceutical composition of claim 73, wherein the solubility enhancer is cyclodextrin.

75. The pharmaceutical composition of claim 54, wherein the inner layer further comprises a rapidly dispersing agent selected from the group of wicking agents, non-effervescent disintegrants, and effervescent disintegrants.

76. The pharmaceutical composition of claim 75, wherein the rapidly dispersing agent is croscarmellose sodium.

77. The pharmaceutical composition of claim 54, wherein upon oral administration of the composition to a mammal, a therapeutically effective amount of the proton pump inhibitor is absorbed across the oral mucosal surface in less than 2 hours.

78. The pharmaceutical composition of claim 77, wherein upon oral administration of the composition to a mammal, a therapeutically effective amount of the proton pump inhibitor is absorbed across the oral mucosal surface in less than 1 hour.

79. The pharmaceutical composition of claim 78, wherein upon oral administration of the composition to a mammal, a therapeutically effective amount of the proton pump inhibitor is absorbed across the oral mucosal surface in less than 45 minutes.

80. The pharmaceutical composition of claim 79, wherein upon oral administration of the composition to a mammal, a therapeutically effective amount of the proton pump inhibitor is absorbed across the oral mucosal surface in less than 30 minutes.

81. The pharmaceutical composition of claim 80, wherein upon oral administration of the composition to a mammal, a therapeutically effective amount of the proton pump inhibitor is absorbed across the oral mucosal surface in less than 15 minutes.

82. The pharmaceutical composition of claim 54, wherein the proton pump inhibitor is in the form of a powder, microspheres, micronized powder, or non-enteric coated microgranules.

83. A method for delivering a therapeutically effective amount of a proton pump inhibitor to a mammal comprising:
applying the pharmaceutical composition of claim 25 to an oral mucosal surface of the mammal; and
allowing a therapeutically effective amount of the proton pump inhibitor to permeate across the mammal's oral mucosal surface into the bloodstream.

84. A method for delivering a therapeutically effective amount of a proton pump inhibitor to a mammal comprising:
applying the pharmaceutical composition of claim 54 to an oral mucosal surface of the mammal; and
allowing a therapeutically effective amount of the proton pump inhibitor to permeate across the mammal's oral mucosal surface into the bloodstream.

85. A method for treating a symptom of a gastric acid disorder in a mammal comprising administering to a mammal the pharmaceutical composition of claim 1.

86. A method for treating a symptom of a gastric acid disorder in a mammal comprising administering to a mammal the pharmaceutical composition of claim 25.

87. A method for treating a symptom of a gastric acid disorder in a mammal comprising administering to a mammal the pharmaceutical composition of claim 54.

88. The pharmaceutical composition of claim 8, wherein the outer layer comprises 0.5-5 grams of the proton pump inhibitor.

89. The pharmaceutical composition of claim 29, wherein the outer layer comprises 0.5-5 grams of the proton pump inhibitor.

90. The pharmaceutical composition of claim 63, wherein the outer layer comprises 0.5-5 grams of the proton pump inhibitor.
